(19) United States
(12) Patent Application Publication
(10) Pub. No.: US 2006/0079680 A1
(43) Pub. Date: Apr. 13, 2006

(54) Pyrazoloazepine compounds as pharmaceutical agents

(75) Inventor: Jason Scott Sawyer, Indianapolis, IN (US)

Correspondence Address: ELI LILLY & COMPANY, PATENT DIVISION, P.O. BOX 6288, INDIANAPOLIS, IN, 46206-6288, US

(73) Assignee: Eli Lilly and Company Patent Division, Indianapolis, IN (US)

(21) Appl. No.: 10/531,111

(22) PCT Filed: Nov. 10, 2003
(86) PCT No.: PCT/US03/32746

§ 371 Date: Apr. 13, 2005

Related U.S. Application Data
(60) Provisional application No. 60/428,892, filed on Nov. 22, 2002.

(51) Int. Cl. C07D 487/04

(52) U.S. Cl. 540/579

(57) ABSTRACT

The disclosed invention relates to compounds of the formula (I), and the pharmaceutically acceptable salts thereof.

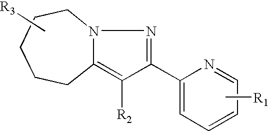

The invention relates to new pyrazoloazepine derivative compounds and their use as pharmaceutical agents, in particular their use as TGF-beta signal transduction inhibitors.

### BACKGROUND OF THE INVENTION

The transforming growth factor-beta (TGF-beta) (“TGF-β”) polypeptides influence growth, differentiation, and gene expression in many cell types. The first polypeptide of this family that was characterized, TGF-β1, has two identical 112 amino acid subunits that are covalently linked. TGF-β1 is a highly conserved protein with only a single amino acid difference distinguishing humans from mice. There are two other members of the TGF-β gene family that are expressed in mammals. TGF-β2 is 71% homologous to TGF-β1 (de Martin, et al. (1987) EMBO J. 6:3673-3677), whereas TGF-β3 is 80% homologous to TGF-β1 (Derynck, et al. (1988) EMBO J 7:3737-3743). There are at least three different extracellular TGF-β receptors, Type I, II and III that are involved in the biological functions of TGF-β1, -β2 and -β3 (For reviews, see Derynck (1994) TIBS 19:548-553 and Massague (1990) Ann. Rev. Cell Biol. 6:597-641). The Type I and Type II receptors are transmembrane serine/threonine kinases which in the presence of TGF-β form a heteromeric signaling complex (Wrana, et al (1992) Cell 71: 1003-1014).

The mechanism of activation of the heteromeric signaling complex at the cell surface has been elucidated (Wrana, et al. (1994) Nature 370: 341-347). TGF-β first binds the type II receptor that is a constitutively active transmembrane serine/threonine kinase. The type I receptor is subsequently recruited into the complex, phoshorylated at the GS domain and activated to phosphorylate downstream signaling components (e.g. Smad proteins) to initiate the intracellular signaling cascade. A constitutively active type I receptor (T204D mutant) has been shown to effectively transduce TGF-β responses, thus bypassing the requirement for TGF-β and the type II receptor (Wieser, et al. (1995) EMBO J 14: 2199-2208). Although no signaling function has been discovered for the type III receptor, it does increase TGF-β2's affinity for the type II receptor making it essentially equipotent with TGF-β1 and TGF-β3 (Lopez-Casillas, et al. (1993) Cell 73: 1435-1444).

Vascular endothelial cells lack the Type III receptor. Instead endothelial cells express a structurally related protein called endoglin (Cheifetz, et al. (1992) J. Biol. Chem. 267:19027-19030), which only binds TGF-β1 and TGF-β3 with high affinity. Thus, the relative potency of the TGF-β's reflects the type of receptors expressed in a cell and organ system. In addition to the regulation of the components in the multi-factorial signaling pathway, the distribution of the synthesis of TGF-β polypeptides also affects physiological function. The distribution of TGF-β2 and TGF-β3 is more limited (Derynck, et al. (1988) EMBO J 7:3737-3743) than TGF-β1, e.g., TGF-β3 is limited to tissues of mesenchymal origin, whereas TGF-β1 is present in both tissues of mesenchymal and epithelial origin.

TGF-β1 is a multifunctional cytokine critical for tissue repair. High concentrations of TGF-β1 are delivered to the site of injury by platelet granules (Assoian and Sporn (1986) J. Cell Biol. 102:1217-1223). TGF-β1 initiates a series of events that promote healing including chemo taxis of cells such as leukocytes, monocytes and fibroblasts, and regulation of growth factors and cytokines involved in angiogenesis, cell division associated with tissue repair and inflammatory responses. TGF-β1 also stimulates the synthesis of extracellular matrix components (Roberts, et al. (1986) Proc. Natl. Acad. Sci. USA 83:4167-4171; Sporn, et al. (1983) Science 219:1329-1330; Massague (1987) Cell 49:437-438) and most importantly for understanding the pathophysiology of TGF-β1, TGF-β1 autoregulates its own synthesis (Kim, et al. (1989) J. Biol. Chem. 264:7041-7045).

The compounds disclosed herein may also exhibit other kinase activity, such as p38 kinase inhibition and/or KDR (VEGFR2) kinase inhibition. Assays to determine such kinase activity are known in the art and one skilled in the art would be able to test the disclosed compounds for such activity.

The compounds disclosed and claimed in this patent application are generally related to compounds disclosed and claimed in PCT patent application number PCT/US002/11884, filed 13 May 2002, which claims priority from U.S. patent application U.S. Ser. No. 60/293,464, filed 24 May 2001, and is herein incorporated by reference.

### SUMMARY OF THE INVENTION

The disclosed invention relates to compounds of the formula: 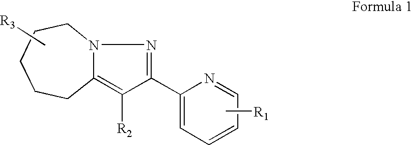
wherein R₁ may be one or more optional substituents selected from the group consisting of: (C1-C6)alkyl, (C2-C6)alkenyl, (C2-C6)alkynyl, (C1-C6)alkoxy, (C2-C6)alkenyloxy, (C2-C6)alkynyloxy, (C1-C6)alkylthio, (C1-C6)alkylsulphinyl, (C1-C6)alkylsulphonyl, (C1-C6)alkylamino, di-[(C1-C6)alkyl]amino, (C1-C6)alkoxycarbonyl, N—(C1-C6)alkylcarbamoyl, N,N-di-[(C1-C6)alkyl]carbamoyl, (C2-C6)alkanoyl, (C2-C6)alkanoyloxy, (C2-C6)alkanoylamino, N—(C1-C6)alkyl-(C2-C6)alkanoylamino, (C3-C6)alkenoylamino, N—(C1-C6)alkyl-(C3-C6)alkenoylamino, (C3-C6)alkynoylamino, N—(C1-C6)alkyl-(C3-C6)alkynoylamino, N—(C1-C6)alkylsulphamoyl, N,N-di-[(C1-C6)alkyl]sulphamoyl, (C1-C6)alkanesulphonylamino, N—(C1-C6)alkyl-(C1-C6)alkanesulphonylamino, carboxamide, ethylene, thiophenyl, aminophenyl, trifluoromethyl, halo, trifluoromethoxy, hydroxymethyl, N-pyrrolidino, N-morpholino, phenylthio, (C1-C4)dialkylaminomethyl, methoxyphenyl, amino, hydroxy, carboxyl, phenyl, arylalkyl;

R₂ is unsubstituted or substituted quinoline; unsubstituted or substituted quinoline N-oxide; unsubstituted or substituted phenyl; unsubstituted or substituted naphthalene; unsubstituted or substituted pyridine; unsubstituted or substituted pyridine N-oxide; unsubstituted or substituted quinazoline; unsubstituted or substituted cinnoline; unsubstituted or substituted benzodioxole; unsubstituted or substituted benzodioxane; unsubstituted or substituted pyrimidine; unsubstituted; substituted benzothiophene; or unsubstituted or substituted phenanthrolene; wherein the substitution may independently be one or more of the following:

(C1-C6)alkyl, (C2-C6)alkenyl, (C2-C6)alkynyl, (C1-C6) alkylhalide, (C1-C6)alkoxy, (C2-C6)alkenyloxy, (C2-C6)alkynyloxy, (C1-C6)alkylthio, (C1-C6)alkylsulphinyl, (C1-C6)alkylsulphonyl, (C1-C6)alkylamino, di-[(C1-C6)alkyl]amino, (C1-C6)alkoxycarbonyl, N—(C1-C6)alkylcarbamoyl, N,N-di-[(C1-C6)alkyl]carbamoyl, aminooxy, N—(C1-C6)alkyl aminooxy, N,N-di-[(C1-C6)alkyl]aminooxy, (C2-C6)alkanoyl, (C2-C6)alkanoyloxy, (C2-C6)alkanoylamino, N—(C1-C6)alkyl-(C2-C6)alkanoylamino, (C3-C6)alkenoylamino, N—(C1-C6)alkyl-(C3-C6)alkenoylaminio, (C3-C6)alkynoylamino, N—(C1-C6)alkyl-(C3-C6)alkynoylamino, sulphamoyl, N—(C1-C6)alkylsulphamoyl, N,N-di-[(C1-C6)alkyl]sulphamoyl, (C1-C6)alkanesulphonylamino, N—(C1-C6)alkyl-(C1-C6)alkanesulphonylamino, carboxamide, ethylene, phenyl, thiophenyl, aminophenyl, phenylthio, halo, cyano, pyridinyl, arylalkyl, hydroxy, N-pyrrolidino, N-morpholino, carboxyl, [5-phenyl-1,2,4-oxadiazole-3-yl]methoxy, 6-methyl-pyridazin-3-yloxy, (5-oxo-2-pyrrolidinyl)methoxy, 2-(4,5-dihydro-1H-imidazolyl), N,N-dialkylcarbamoyloxy, 1-hydroxy-1-methylethyl, 4-fluorophenyl, 3,4-methylenedioxyphenyl, trifluoromethyl, trifluoromethoxy,
or a group of the formula: 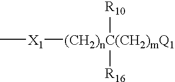
wherein: X₁ is O, N, S, SO₂, NR₁₃, C(O), or bond; Q₁ is hydrogen, phenyl, 5-(2,2-difluoro-1,3-benzodioxolyl), C(O)Q₅, or pyridyl when, except when one is 0 the other cannot be 0 are independently 0-2; Q₁ is OR₁₁, NR₁₁R₁₂, halo, N-morpholino, N-piperazino-N′R₁₃, N-imidazolyl, N-pyrazolyl, N-triazolyl, N-(4-piperidinylpiperidine), SO₂R₁₄, SOR₁₄, NHSO₂R₁₅, acetamnido, N-phthalimido, N-oxazolidino, N-imidazolino, N-benzoxazolidino, N-pyrolidinonyl, N(N′-methylbenzimidazolino), N,N-di(C1-C4)alkylamino(C1-C4)alkoxy, N-benzimidazolino; when m and n are independently 0-2, but one or the other of m or n is not 0; Q₅ is hydroxy, methoxy, amino, diethylamino, dimethylamino; R₁₀ is hydrogen, halo, (C1-C6)alkyl; R₁₁ and R₁₂ are independently hydrogen, (C1-C6)alkyl, (C1-C6)alkoxy, arylalkyl, (C3-C8)cycloalkyl, (C3-C8)cycloalkylmethyl, 4-(N-methylpiperidinyl), pyridyl, or R₁₁ and R₁₀ can be taken together to form a 4, 5, 6, or 7 membered ring, or R₁₁ and R₁₂ can be taken together to form a 3, 4, 5, 6, or 7 membered ring; R₁₃ is hydrogen, (C1-C6)alkyl, 2-methoxyphenyl, 2-pyridimidinyl; R₁₄ is 2-pyrimidinyl, N-methyl-2-imidazolyl, 4-chlorophenyl, 2-pyridylmethyl; R₁₅ is (C1-C6)alkyl, N-methyl-4-imidazolyl; R₁₆ is hydrogen, halo, arylalkyl, aryl,
or a group of the formula: 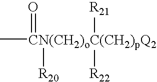
wherein: Q₂ is hydrogen, 4-imidazolyl, or C(O)NR₂₄R₂₅ when o and p are independently 0-2; Q₂ is OR₂₃, NR₂₄R₂₅, or N-morpholino, when o and p are independently 0-2, but one or the other of o or p is not 0; R₂₀ is hydrogen, or (C1-C6)alkyl; R₂₁ is hydrogen, (C1-C6)alkyl, or R₂₁ and R₂₀ can be taken together to form a 4, 5, 6, or 7 membered ring; R₂₂ is hydrogen, (C1-C6)alkyl, arylalkyl, aryl, or R₂₁ and R₂₂ can be taken together to be a 3, 4, 5, 6, 7 membered ring; R₂₃ is hydrogen or (C1-C6)alkyl; R₂₄ is hydrogen, (C1-C6)alkyl, or R₂₄ and R₂₅ can be taken together to form a 3, 4, 5, 6, or 7 membered ring, or R₂₄ and R₂₀ can be taken together to form a 6 or 7 membered ring; R₂₅ is hydrogen, (C1-C6)alkyl, or acetyl,
or a group of the formula: 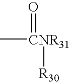
wherein: R₃₀ is hydrogen, or (C1-C6)alkyl; R₃₁ is hydrogen, (C1-C6)alkyl, 2-pyridyl, pyridylmethyl, amino, or hydroxy,
or a group of the formula:
—NR₃₂R₃₃
wherein: R₃₂ and R₃₃ are each independently hydrogen, (C1-C6)alkyl, acetyl, (C1-C4)alkylsulphonyl, or R₃₂ and R₃₃ can be taken together to form a 4, 5, 6, or 7 membered ring,
or a group of the formula: 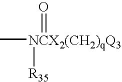
wherein: X₂ is CH₂, O, or N; q is 2-3 except when Q₃ is a bond, q is 0-3; Q₃ is NR₃₆R₃₇, or OR₃₈, and R₃₅ is hydrogen, or R₃₅ and Q₃ can be taken together to form a 5 membered ring; R₃₆, R₃₇, and R₃₈ are each independently hydrogen, or (C1-C6)alkyl,
or a group of the formula: 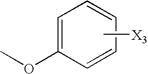
wherein: X₃ is cyano, carboxamide, N,N-dimethylcarboxamide, N,N-dimethylthiocarboxamide, N,N-dimethylaminomethyl, 4-methylpiperazin-1yl-methyl or carboxylate,
or a group of the formula: 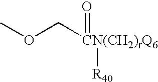
wherein: Q₆ is NR₄₁R₄₂; r is 2-3; R₄₀ is hydrogen, or (C1-C6)alkyl; R₄₁, and R₄₂ are hydrogen, (C1-C6)alkyl, or R₄, and R₄₀ can be taken together to form a 6 or 7 membered ring,
or a group of the formula: 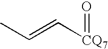
wherein: Q₇ is hydroxy, methoxy, dimethylamino, or N-piperidinyl;

R₃ may be one or more optional substituents selected from the group consisting of (C1-C6 alkyl);

and the pharmaceutically acceptable salts thereof.

Preferred compounds of the invention are compounds: 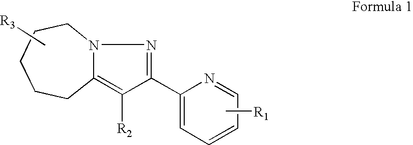
wherein R₁ may be one or more optional substituents selected from the group consisting of: (C1-C6)alkyl, (C2-C6)alkenyl, (C2-C6)alkynyl, (C1-C6)alkoxy, (C2-C6)alkenyloxy, (C2-C6)alkynyloxy, (C1-C6)alkylthio, (C1-C6)alkylsulphinyl, (C1-C6)alkylsulphonyl, (C1-C6)alkylamino, di-[(C1-C6)alkyl]amino, (C1-C6)alkoxycarbonyl, N—(C1-C6)alkylcarbamoyl, N,N-di-[(C1-C6)alkyl]carbamoyl, (C2-C6)alkanoyl, (C2-C6)alkanoyloxy, (C2-C6)alkanoylamino, N—(C1-C6)alkyl-(C2-C6)alkanoylamino, (C3-C6)alkenoylamino, N—(C1-C6)alkyl-(C3-C6)alkenoylamino, (C3-C6)alkynoylamino, N—(C1-C6)alkyl-(C3-C6)alkynoylamino, N—(C1-C6)alkylsulphamoyl, N,N-di-[(C1-C6)alkyl]sulphamoyl, (C1-C6)alkanesulphonylamino, N—(C1-C6)alkyl-(C1-C6)alkanesulphonylamino, carboxamide, ethylene, thiophenyl, aminophenyl, trifluoromethyl, halo, trifluoromethoxy, hydroxymethyl, N-pyrrolidino, N-morpholino, phenylthio, (C1-C4)dialkylaminomethyl, methoxyphenyl, amino, hydroxy, carboxyl, phenyl, arylalkyl;

R₂ is unsubstituted or substituted quinoline; unsubstituted or substituted quinoline N-oxide; substituted or unsubstituted quinazoline; substituted or unsubstituted pyrimidine; wherein the substitution may independently be one or more of the following: (C1-C6)alkyl, (C2-C6)alkenyl, (C2-C6)alkynyl, (C1-C6) alkylhalide, (C1-C6)alkoxy, (C2-C6)alkenyloxy, (C2-C6)alkynyloxy, (C1-C6)alkylthio, (C1-C6)alkylsulphinyl, (C1-C6)alkylsulphonyl, (C1-C6)alkylamino, di-[(C1-C6)alkyl]amino, (C1-C6)alkoxycarbonyl, N—(C1-C6)alkylcarbamoyl, N,N-di-[(C1-C6)alkyl]carbamoyl, aminooxy, N—(C1-C6)alkyl aminooxy, N,N-di-[(C1-C6)alkyl]aminooxy, (C2-C6)alkanoyl, (C2-C6)alkanoyloxy, (C2-C6)alkanoylamino, N—(C1-C6)alkyl-(C2-C6)alkanoylamino, (C3-C6)alkenoylamino, N—(C1-C6)alkyl-(C3-C6)alkenoylamino, (C3-C6)alkynoylamino, N—(C1-C6)alkyl-(C3-C6)alkynoylamino, sulphamoyl, N—(C1-C6)alkylsulphamoyl, N,N-di-[(C1-C6)aLkyl]sulphamoyl, (C1-C6)alkanesulphonylamino, N—(C1-C6)alkyl-(C1-C6)alkanesulphonylamino, carboxamide, ethylene, phenyl, thiophenyl, aminophenyl, phenylthio, halo, cyano, pyridinyl, arylalkyl, hydroxy, N-pyrrolidino, N-morpholino, carboxyl, [5-phenyl-1,2,4-oxadiazole-3-yl]methoxy, 6-methyl-pyridazin-3-yloxy, (5-oxo-2-pyrrolidinyl)methoxy, 2-(4,5-dihydro-1H-imidazolyl), N,N-dialkylcarbamoyloxy, 1-hydroxy-1-methylethyl, 4-fluorophenyl, 3,4-methylenedioxyphenyl, trifluoromethyl, trifluoromethoxy,
or a group of the formula: 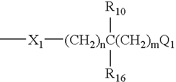
wherein: X₁ is O, N, S, SO₂, NR₁₃, C(O), or bond; Q₁ is hydrogen, phenyl, 5-(2,2-difluoro-1,3-benzodioxolyl), C(O)Q₅, or pyridyl when, except when one is 0 the other cannot be 0 are independently 0-2; Q₁ is OR₁₁, NR₁₁R₁₂, halo, N-morpholino, N-piperazino-N′R₁₃, N-imidazolyl, N-pyrazolyl, N-triazolyl, N-(4-piperidinylpiperidine), SO₂R₁₄, SOR₁₄, NHSO₂R₁₅, acetamido, N-phthalimido, N-oxazolidino, N-imidazolino, N-benzoxazolidino, N-pyrolidinonyl, N(N′-methylbenzimidazolino), N,N-di(C1-C4)alkylamino(C1-C4)alkoxy, N-benzimidazolino; when m and n are independently 0-2, but one or the other of m or n is not 0; Q₅ is hydroxy, methoxy, amino, diethylamino, dimethylamino; R₁₀ is hydrogen, halo, (C1-C6)alkyl; R₁₁ and R₁₂ are independently hydrogen, (C1-C6)alkyl, (C1-C6)alkoxy, arylalkyl, (C3-C8)cycloalkyl, (C3-C8)cycloalkylmethyl, 4-(N-methylpiperidinyl), pyridyl, or R₁₁ and R₁₀ can be taken together to form a 4, 5, 6, or 7 membered ring, or R₁₁ and R₁₂ can be taken together to form a 3, 4, 5, 6, or 7 membered ring; R₁₃ is hydrogen, (C1-C6)alkyl, 2-methoxyphenyl, 2-pyridimidinyl; R₁₄ is 2-pyrimidinyl, N-methyl-2-imidazolyl, 4-chlorophenyl, 2-pyridylmethyl; R₁₅ is (C1-C6)alkyl, N-methyl-4-imidazolyl; R₁₆ is hydrogen, halo, arylalkyl, aryl,
or a group of the formula: 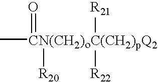
wherein: Q₂ is hydrogen, 4-imidazolyl, or C(O)NR₂₄R₂₅ when o and p are independently 0-2; Q₂ is OR₂₃, NR₂₄R₂₅, or N-morpholino, when o and p are independently 0-2, but one or the other of o or p is not 0; R₂₀ is hydrogen, or (C1-C6)alkyl; R₂₁ is hydrogen, (C1-C6)alkyl, or R₂₁ and R₂₀ can be taken together to form a 4, 5, 6, or 7 membered ring; R₂₂ is hydrogen, (C1-C6)alkyl, arylalkyl, aryl, or R₂₁ and R₂₂ can be taken together to be a 3, 4, 5, 6, 7 membered ring; R₂₃ is hydrogen or (C1-C6)alkyl; R₂₄ is hydrogen, (C1-C6)alkyl, or R₂₄ and R₂₅ can be taken together to form a 3, 4, 5, 6, or 7 membered ring, or R₂₄ and R₂₀ can be taken together to form a 6 or 7 membered ring; R₂₅ is hydrogen, (C1-C6)alkyl, or acetyl,
or a group of the formula: 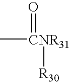
wherein: R₃₀ is hydrogen, or (C1-C6)alkyl; R₃₁ is hydrogen, (C1-C6)alkyl, 2-pyridyl, pyridylmethyl, amino, or hydroxy,
or a group of the formula:
—NR₃₂R₃₃
wherein: R₃₂ and R₃₃ are each independently hydrogen, (C1-C6)alkyl, acetyl, (C1-C4)alkylsulphonyl, or R₃₂ and R₃₃ can be taken together to form a 4, 5, 6, or 7 membered ring,
or a group of the formula: 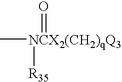
wherein: X₂ is CH₂, O, or N; q is 2-3 except when Q₃ is a bond, q is 0-3; Q₃ is NR₃₆R₃₇, or OR₃₈, and R₃₅ is hydrogen, or R₃₅ and Q₃ can be taken together to form a 5 membered ring; R₃₆, R₃₇, and R₃s are each independently hydrogen, or (C1-C6)alkyl,
or a group of the formula: 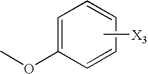
wherein: X₃ is cyano, carboxamide, N,N-dimethylcarboxamide, N,N-dimethylthiocarboxamide, N,N-dimethylaminomethyl, 4-methylpiperazin-1yl-methyl or carboxylate,
or a group of the formula: 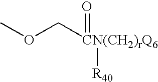
wherein: Q₆ is NR₄₁R₄₂; r is 2-3; R₄₀ is hydrogen, or (C1-C6)alkyl; R₄₁ and R₄₂ are hydrogen, (C1-C6)alkyl, or R₄₁ and R₄₀ can be taken together to form a 6 or 7 membered ring,
or a group of the formula: 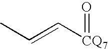
wherein: Q₇ is hydroxy, methoxy, dimethylamino, or N-piperidinyl;

R₃ may be one or more optional substituents selected from the group consisting of (C1-C6 alkyl);

and the pharmaceutically acceptable salts thereof.

More preferred compounds of the invention are the compounds: 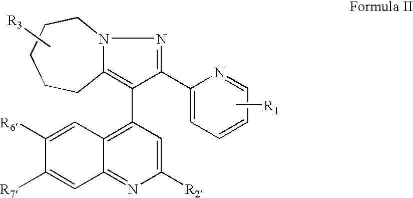
wherein R1 may be one or more optional substituents selected from the group consisting of: (C1-C6)alkyl, (C2-C6)alkenyl, (C2-C6)alkynyl, (C1-C6)alkoxy, (C2-C6)alkenyloxy, (C2-C6)alkynyloxy, (C1-C6)alkylthio, (C1-C6)alkylsulphinyl, (C1-C6)alkylsulphonyl, (C1-C6)alkylamino, di-[(C1-C6)alkyl]amino, (C1-C6)alkoxycarbonyl, N—(C1-C6)alkylcarbamoyl, N,N-di-[(C1-C6)alkyl]carbamoyl, (C2-C6)alkanoyl, (C2-C6)alkanoyloxy, (C2-C6)alkanoylamino, N—(C1-C6)alkyl-(C2-C6)alkanoylamino, (C3-C6)alkenoylamino, N—(C1-C6)alkyl-(C3-C6)alkenoylamino, (C3-C6)alkynoylamino, N—(C1-C6)alkyl-(C3-C6)alkynoylamino, N—(C1-C6)alkylsulphamoyl, N,N-di-[(C1-C6)alkyl]sulphamoyl, (C1-C6)alkanesulphonylamino, N—(C1-C6)alkyl-(C1-C6)alkanesulphonylamino, carboxamide, ethylene, thiophenyl, aminophenyl, trifluoromethyl, halo, trifluoromethoxy, hydroxymethyl, N-pyrrolidino, N-morpholino, phenylthio, dialkylaminomethyl, methoxyphenyl, amino, hydroxy, carboxyl, phenyl, arylalkyl;

R2′ is hydrogen; (C1-C6)alkyl; (C1-C6)alkylthio; (C1-C6)alkoxy; halo; thiophenyl; aminophenyl; N-pyrrolidino; N-morpholino;

R6′ and R7′ are independently one or more of the following: hydrogen, (C1-C6)alkyl, (C2-C6)alkenyl, (C2-C6)alkynyl, (C1-C6) alkylhalide, (C1-C6)alkoxy, (C2-C6)alkenyloxy, (C2-C6)alkynyloxy, (C1-C6)alkylthio, (C1-C6)alkylsulphinyl, (C1-C6)alkylsulphonyl, (C1-C6)alkylamino, di-[(C1-C6)alkyl]amino, (C1-C6)alkoxycarbonyl, N—(C1-C6)alkylcarbamoyl, N,N-di-[(C1-C6)alkyl]carbamoyl, aminooxy, N—(C1-C6)alkyl aminooxy, N,N-di-[(C1-C6)alkyl]aminooxy, (C2-C6)alkanoyl, (C2-C6)alkanoyloxy, (C2-C6)alkanoylamino, N—(C1-C6)alkyl-(C2-C6)alkanoylamino, (C3-C6)alkenoylamino, N—(C1-C6)alkyl-(C3-C6)alkenoylamino, (C3-C6)alkynoylamino, N—(C1-C6)alkyl-(C3-C6)alkynoylamino, sulphamoyl, N—(C1-C6)alkylsulphamoyl, N,N-di-[(C1-C6)alkyl]sulphamoyl, (C1-C6)alkanesulphonylamino, N—(C1-C6)alkyl-(C1-C6)alkanesulphonylamino, carboxamide, ethylene, phenyl, thiophenyl, aminophenyl, phenylthio, halo, cyano, pyridinyl, arylalkyl, hydroxy, N-pyrrolidino, N-morpholino, carboxyl, [5-phenyl-1,2,4-oxadiazole-3-yl]methoxy, 6-methyl-pyridazin-3-yloxy, (5-oxo-2-pyrrolidinyl)methoxy, 2-(4,5-dihydro-1H-imidazolyl), N,N-dialkylcarbamoyloxy, 1-hydroxy-1-methylethyl, 4-fluorophenyl, 3,4-methylenedioxyphenyl, trifluoromethyl, trifluoromethoxy,
or a group of the formula 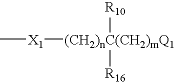
wherein: X₁ is O, N, S, SO₂, NR₁₃, C(O), or bond; Q₁ is hydrogen, phenyl, 5-(2,2-difluoro-1,3-benzodioxolyl), C(O)Q₅, or pyridyl when m and n are independently 0-2, except when one is 0 the other cannot be 0; Q₁ is OR₁₁, NR₁₁R₁₂, halo, N-morpholino, N-piperazino-N′R₁₃, N-imidazolyl, N-pyrazolyl, N-triazolyl, N-(4-piperidinylpiperidine), SO₂R₁₄, SOR₁₄, NHSO₂R₁₅, acetamido, N-phthalimido, N-oxazolidino, N-imidazolino, N-benzoxazolidino, N-pyrolidinonyl, N(N′-methylbenzimidazolino), N,N-di(C1-C4)alkylamino(C1-C4)alkoxy, N-benzimidazolino; when m and n are independently 0-2, but one or the other of m or n is not 0; Q₅ is hydroxy, methoxy, amino, diethylamino, dimethylamino; R₁₀ is hydrogen, halo, (C1-C6)alkyl; R₁₁ and R₁₂ are independently hydrogen, (C1-C6)alkyl, (C1-C6)alkoxy, arylalkyl, cycloalkyl, cycloalkylmethyl, 4-(N-methylpiperidinyl), pyridyl, or R₁₁ and R₁₀ can be taken together to form a 4, 5, 6, or 7 membered ring, or R₁₁ and R₁₂ can be taken together to form a 3, 4, 5, 6, or 7 membered ring; R₁₃ is hydrogen, (C1-C6)alkyl, 2-methoxyphenyl; R₁₄ is 2-pyrimidinyl, N-methyl-2-imidazolyl, 4-chlorophenyl, 2-pyridylmethyl; R₁₅ is (C1-C6)alkyl, N-methyl-4-imidazolyl; R₁₆ is hydrogen, halo, arylalkyl, aryl,
or a group of the formula: 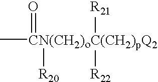
wherein: Q₂ is hydrogen, 4-imidazolyl, or C(O)NR₂₄R₂₅ when o and p are independently 0-2; Q₂ is OR₂₃, NR₂₄R₂₅, or N-morpholino, when o and p are independently 0-2, but one or the other of o or p is not 0; R₂₀ is hydrogen, or (C1-C6)alkyl; R₂₁ is hydrogen, (C1-C6)alkyl, or R₂₁ and R₂₀ can be taken together to form a 4, 5, 6, or 7 membered ring; R₂₂ is hydrogen, (C1-C6)alkyl, arylalkyl, aryl, or R₂₁ and R₂₂ can be taken together to be a 3, 4, 5, 6, 7 membered ring; R₂₃ is hydrogen or (C1-C6)alkyl; R₂₄ is hydrogen, (C1-C6)alkyl, or R₂₄ and R₂₅ can be taken together to form a 3, 4, 5, 6, or 7 membered ring, or R₂₄ and R₂₀ can be taken together to form a 6 or 7 membered ring; R₂₅ is hydrogen, (C1-C6)alkyl, or acetyl,
or a group of the formula: 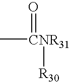
wherein: R₃₀ is hydrogen, or (C1-C6)alkyl; R₃₁ is hydrogen, (C1-C6)alkyl, 2-pyridyl, pyridylmethyl, amino, or hydroxy,
or a group of the formula:
—NR₃₂R₃₃
wherein: R₃₂ and R₃₃ are each independently hydrogen, (C1-C6)alkyl, acetyl, alkylsulphonyl, or R₃₂ and R₃₃ can be taken together to form a 4, 5, 6, or 7 membered ring,
or a group of the formula: 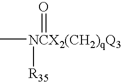
wherein: X₂ is CH₂, O, or N; q is 2-3 except when Q₃ is a bond, q is 0-3; Q₃ is NR₃₆R₃₇, OR₃₈, or a bond; R₃₅ is hydrogen, or R₃₅ and Q₃ (when Q₃ is a bond) can be taken together to form a 5 membered ring; R₃₆, R₃₇, and R₃₈ are each independently hydrogen, or (C1-C6)alkyl,
or a group of the formula: 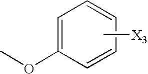
wherein: X₃ is cyano, carboxamide, N,N-dimethylcarboxamide, N,N-dimethylthiocarboxamide, N,N-dimethylaminomethyl, 4-methylpiperazin-1yl-methyl or carboxylate,
or a group of the formula: 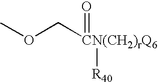
wherein: Q₆ is NR₄₁R₄₂; r is 2-3; R₄₀ is hydrogen, or (C1-C6)alkyl; R₄₁, and R₄₂ are hydrogen, (C1-C6)alkyl, or R₄₁ and R₄₀ can be taken together to form a 6 or 7 membered ring,
or a group of the formula: 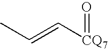
wherein: Q₇ is hydroxy, methoxy, dimethylamino, or N-piperidinyl;

R₃ may be one or more optional substituents selected from the group consisting of (C1-C6 alkyl);

and the pharmaceutically acceptable salts thereof.

Another more preferred embodiment of the invention include compounds of the formula: 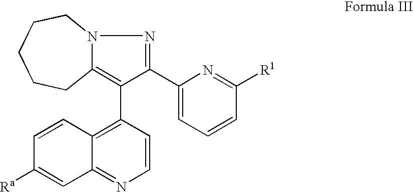
wherein
R¹ is hydrogen or methyl;
R^{a} is hydroxy; (C1-C4)alkoxy, or —O(CH₂)₂N-morpholino;
and the pharmaceutically acceptable salts thereof.

### DETAILED DESCRIPTION OF THE INVENTION

The term “effective amount” as used in “an effective amount of a compound of Formula I,” for example, refers to an amount of a compound of the present invention that is capable of inhibiting TGF beta.

The general chemical terms used herein have their usual meanings.. For example, as used herein, the term “C₁-C₄ alkyl”, alone or in combination, denotes a straight-chain or branched-chain C₁-C₄ alkyl group consisting of carbon and hydrogen atoms, examples of which are methyl, ethyl, propyl, isopropyl, butyl, sec-butyl, tert-butyl, and the like. The term “C₃-C₆ cycloalkyl” refers to cyclopropyl, cyclobutyl, cyclopentyl, cyclohexyl.

The term “C₁-C₄ alkoxy”, alone or in combination, denotes an alkyl group as defined earlier, which is attached via an oxygen atom, such as, for example, methoxy, ethoxy, propoxy, isopropoxy, butoxy, tert-butoxy, and the like. The term “C₁-C₄ alkylthio”, alone or in combination, denotes an alkyl group as defined earlier and is attached via a sulfur atom, and includes methylthio, ethylthio, isobutylthio, and the like.

As used herein, the term “halo” or “halogen” represents fluorine, chlorine, bromine, or iodine. The term “hydroxy,” alone or in combination, represents an —OH moiety. The term “carboxy” or “carboxyl” refers to a carboxylic acid. The term “carboxamide” refers to a carbonyl substituted with an —NH₂ moiety. The term “oxo” refers to a carbonyl group.

As used herein, the term “aryl” represents a substituted or unsubstituted phenyl or naphthyl. Aryl may be optionally substituted with one or more groups independently selected from hydroxy, carboxy, C₁-C₆ alkoxy, C₁-C₆ alkyl, halogen, carboxamide, trifluoromethyl, hydroxymethyl, and hydroxy(C₁-C₄)alkyl.

The term “C₃-C₈ cycloalkyl” refers to cyclopropyl, cyclobutyl, cyclopentyl, cyclohexyl, cycloheptyl, and cyclooctyl. The term “optionally substituted C₃-C₈ cycloalkyl” refers to a C₃-C₈ cycloalkyl as defined herein unsubstituted or substituted with one or more groups independently selected from hydroxy, carboxy, C₁₋₆ alkoxy, C₁₋₆ alkyl, halogen, carboxamide, trifluoromethyl, hydroxymethyl, and hydroxy(C₁-C₄)alkyl.

As used herein, the term “C₁-C₆ alkyl” refers to straight or branched, monovalent, saturated aliphatic chains of 1 to 6 carbon atoms and includes, but is not limited to, methyl, ethyl, propyl, isopropyl, butyl, isobutyl, t-butyl, pentyl, isopentyl, and hexyl. The term “C₁-C₆ alkyl” includes within its definition the terms “C₁-C₄ alkyl” and “C₁-C₃ alkyl.”

“C₁-C₆ alkenyl” refers to a straight or branched, divalent, unsaturated aliphatic chain of 1 to 6 carbon atoms and includes, but is not limited to, methylenyl, ethylenyl, propylenyl, isopropylenyl, butylenyl, isobutylenyl, t-butylenyl, pentylenyl, isopentylenyl, hexylenyl.

“C₁-C₆ alkoxycarbonyl” represents a straight or branched C₁-C₆ alkoxy chain, as defined above, that is attached via the oxygen atom to a carbonyl moiety. Typical C₁-C₆ alkoxycarbonyl groups include methoxycarbonyl, ethoxycarbonyl, propoxycarbonyl, isopropoxycarbonyl, butoxycarbonyl, t-butoxycarbonyl and the like.

The term “di(C₁-C₆ alkyl)amino” refers to a group of the formula: 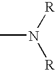
wherein each R group independently represents a “C₁-C₆ alkyl” group, as defined above.

An “optionally substituted phenyl” is a phenyl ring that is unsubstituted or substituted with 1 to 5 substituents, more preferably 1 to 3 substituents, for example: halo, C₁-C₆ alkyl, C₁-C₆ alkoxy, C1-C6 alkylamino, trifluoromethyl, nitro, and cyano.

An “optionally substituted benzyl” is a benzyl ring that is unsubstituted or substituted with 1 to 5 substituents, more preferably 1 to 3 substituents, for example: halo, C₁-C₆ alkyl, C₁-C₆ alkoxy, trifluoromethyl, nitro, and cyano.

“Phenoxycarbonyl” refers to the group: phenyl-O—C(O)—. “Aryl” refers to an unsaturated aromatic carbocyclic group of 6 to 14 carbon atoms having a single ring (e.g., phenyl) or multiple condensed (fused) rings (e.g., naphthyl or anthracenyl).

Unless otherwise constrained by the definition for the aryl substituent, such aryl groups can optionally be substituted with 1 to 5 substituents, more preferably 1 to 3 substituents, selected from the group consisting of halo, hydroxy, acetyl, nitro, cyano, C₁-C₆ alkyl, C₁-C₆ alkoxy, phenyl, di(C₁-C₆ alkyl)amino, trifluoromethyl, trifluoromethoxy, —S(O)ₘ—(C₁-C₆ alkyl), and —S(O)ₘ-(phenyl), wherein m can be 0, 1, or 2.

“Arylalkyl” refers to aryl groups attached to alkyl groups, preferably having 1 to 6 carbon atoms in the alkyl moiety and 6 to 10 carbon atoms in the aryl moiety. Such arylalkyl groups are exemplified by benzyl, phenethyl, and the like.

Unless otherwise constrained by the definition for arylalkyl, such arylalkyl groups can be optionally substituted with 1 to 5 substituents, more preferably 1 to 3 substituents, selected from the group consisting of halo, hydroxy, nitro, cyano, C₁-C₆ alkyl, C₁-C₆ alkoxy, di(C₁-C₆ alkyl)amino, trifluoromethyl, trifluoromethoxy, carbamoyl, pyrrolidinyl, —S(O)ₘ—(C₁-C₆ alkyl), and —S(O)ₘ-(phenyl), wherein m can be 0, 1, or 2. The arylalkyl groups may be optionally substituted on the aryl moiety, the alkyl moiety, or both the aryl moiety and the alkyl moiety.

### Compounds Exemplified in the Application Include the Following

a. 3-quinolin-4-yl-2-pyridin-2-yl-5,6,7,8-tetrahydro-4H-pyrazolo[1,5-a]azepine.
b. (7-Methoxy-quinolin-4-yl)-2-pyridin-2-yl-5,6,7,8-tetrahydro-4H-pyrazolo[1,5-a]azepine.
c. 4-(2-Pyridin-2-yl-5,6,7,8-tetrahydro-4H-pyrazolo[1,5-a]azepin-3-yl)-quinolin-7-ol.
d. 3-[7-(2-Morpholin-4-yl-ethoxy)-quinolin-4-yl]-2-pyridin-2-yl-5,6,7,8-tetrahydro-4H-pyrazolo[1,5-a]azepine.

The compounds exemplied above are merely representative of the invention and are not limiting in any fashion.

The compounds disclosed herein can be made according to the following schemes and examples. The examples should in no way be understood to be limiting in any way as to how the compounds may be made.

The skilled artisan will appreciate that the introduction of certain substituents will create asymmetry in the compounds of Formula (I). The present invention contemplates all enantiomers and mixtures of enantiomers, including racemates. It is preferred that the compounds of the invention containing chiral centers are single enantiomers.

The compounds of the present invention can be prepared by a variety of procedures, some of which are illustrated in the Schemes below. It will be recognized by one of skill in the art that the individual Steps in the following schemes may be varied to provide the compounds of Formula (I). The particular order of Steps required to produce the compounds of Formula (I) is dependent upon the particular compound being synthesized, the starting compound, and the relative lability of the substituted moieties. Additional analogous methods are described in PCT patent application number PCT/US002/11884, filed 13 May 2002, incorporated by reference herein.

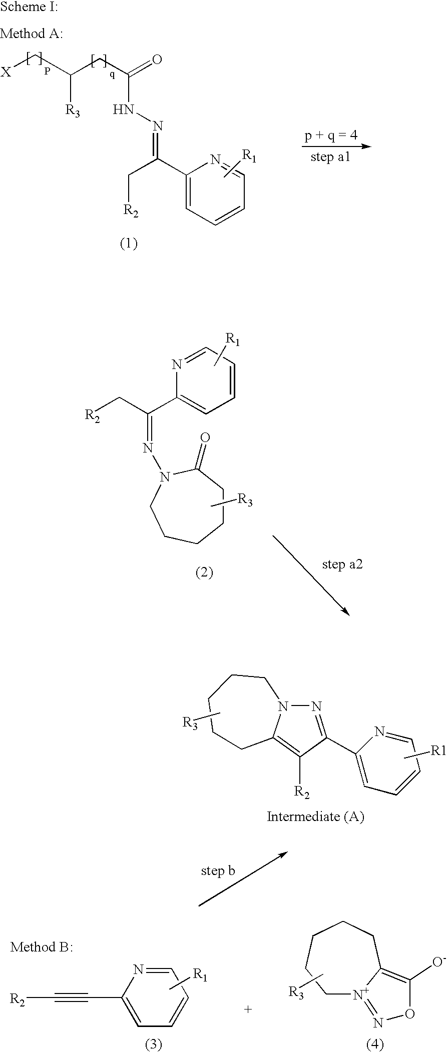

In Scheme I, Step a1 depicts the cyclization of a compound of formula (1), where the R group(s) can be any group(s), previously defined as said for R₁, R₂ or R₃ of Formula (I) from here on. Typically, the appropriate compound of formula (1) is contacted to a suitable base that can form the anion of the hydrazone, such as lithium diisopropylamide, potassium bis(trimethylsilyl)amide, lithium bis(trimethylsilyl)amide, sodium bis(trimethylsilyl)amide, cesium carbonate, sodium hydride, lithium hydride, potassium hydride, sodium alkoxides (sodium hydoxide, sodium methoxide, or sodium ethoxide) or potassium alkoxides (potassium hydroxide, potassium methoxide, or potassium ethoxide), with sodium hydride being the preferred base. The reaction is carried out in a suitable solvent, such as tetrahydrofuran, N,N-dimethylformamide, N-methylpyrolidin-2-one, dimethylsulfoxide or toluene, preferably tetrahydrofuran, at temperatures of about 0 to 100° C. Step a2 depicts the cyclization of a compound of formula (2). Typically, the appropriate compound of formula (2) is contacted to a suitable base that can form the anion of the hydrazone, such as lithium diisopropylamide, potassium bis(trimethylsilyl)amide, lithium bis(trimethylsilyl)amide, sodium bis(trimethylsilyl)amide, cesium carbonate, sodium hydride, lithium hydride, potassium hydride, sodium alkoxides (sodium hydoxide, sodium methoxide, or sodium ethoxide) or potassium alkoxides (potassium hydroxide, potassium methoxide, or potassium ethoxide), with cesium carbonate being the preferred base. The reaction is carried out in a suitable solvent, such as tetrahydrofuran, N,N-dimethylformamide, N-methylpyrolidin-2-one, dimethylsulfoxide or toluene, preferably N-methylpyrolidin-2-one at temperatures of about 0 to 100° C. The reaction conditions for Step a2 are the same. The products can be isolated and purified by techniques well known in the art, such as precipitation, filtration, extraction, evaporation, trituration, chromatography, and recrystallization.

Another variation a skilled artisan would appreciate is Method B for the formation of Intermediate (A), in Scheme I, is Step b, which is known and appreciated in the art (Ranganathan, Darshan; Bamezai, Shakti, Tetrahedron Lett., 1983, 1067-1070). For example, an alkyne (3) is reacted with a compound (4) in a suitable solvent, such as tetrahydrofuran, N,N-dimethylformamide, or toluene, xylene, preferably xylene at temperatures of about 0 to 150° C. The products can be isolated and purified by techniques described above.

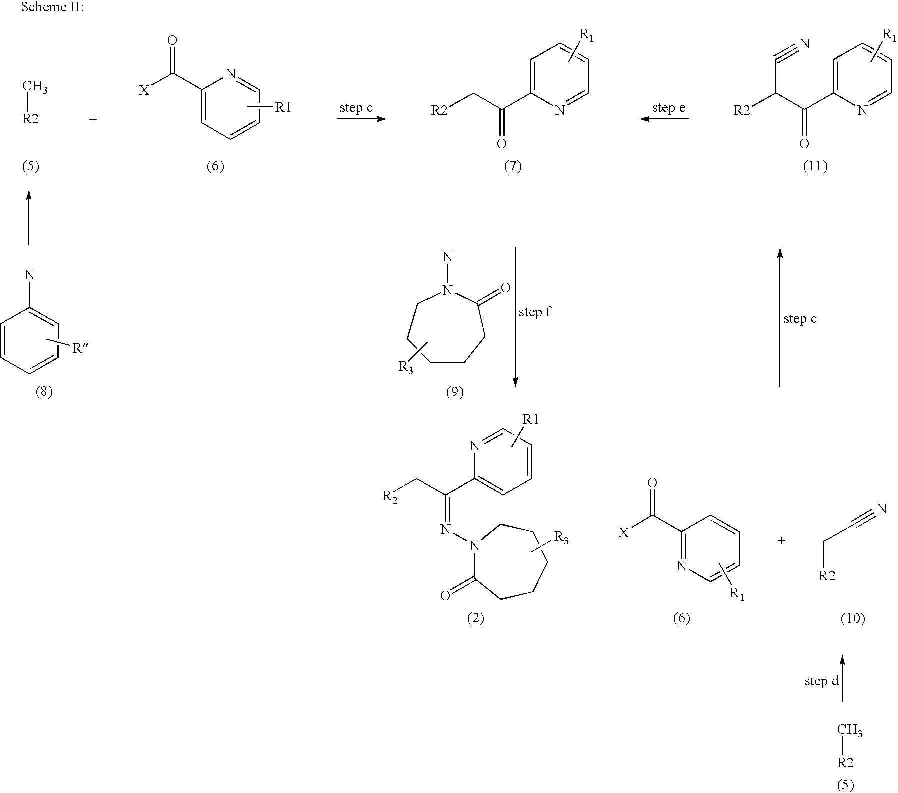

Scheme II, Step c, depicts an acylation of an appropriate R₂ group on a compound of formula (5) and an appropriate carbonyl ester of formula (6) to give a compound of formula (7). The compounds of formula (5) are commercially available or can be produced by a condensation-cyclization by the use of an appropriate substituted aryl-heteroaryl-amine of formula (8), where R″ is previously described as substitutions for the R2 groups of Formula (I). For an example, methyl vinyl ketone can be reacted with formula (8) in the presence of an acid to afford aromatic-heteroaromatic-methyl compounds of formula (5). The acylation of formula (5) requires that X, of formula (6), be a suitable leaving group, such as C1-C6 alkoxy, disubstituted amino, halo, C1-C6 thioether, preferably C1-C6 alkoxy. The reaction is typically carried out in the presence of a suitable base that can create an anion of the compound of formula (5), such as lithium diisopropylamide, potassium bis(trimethylsilyl)amide, lithium bis(trimethylsilyl)amide, sodium bis(trimethylsilyl)amide, sodium hydride, lithium hydride, potassium hydride, sodium alkoxides (sodium methoxide, or sodium ethoxide) or potassium alkoxides (potassium methoxide, or potassium ethoxide), with potassium bis(trimethylsilyl)amide being the preferred base. Generally, the reaction is carried out in suitable solvents, such as tetrahydrofuran and toluene or a combination of such, at temperatures of about −78° C. to ambient temperature. The product, formula (7), can be isolated and purified by techniques well known in the art, such as precipitation, filtration, extraction, evaporation, trituration, chromatography, and recrystallization. Another variation of the acylation Step c, is to use a nitrile compound of formula (10) in place of the compound of formula (5). The product, formula (11), can be transformed to formula (7) by hydrolysis of the nitrile group and then subsequent decarboxylation. Generally, a compound of formula (11) is dissolved in a hydrogen halide acid solution, preferably hydrogen chloride. The reaction is carried out at temperatures of about ambient to refluxing for about 24 hours. This type of reaction is well known and appreciated in the art (Larock, R. C., Comprehensive Organic Transformations, copyright 1989, VCH, pp 993). Compounds of formula (10) can be acquired by treatment of an appropriate substituted aromatic- or heteroaromatic-methyl group with a halogenating reagent, such as N-halosuccinimides, preferably N-bromosuccinimide in carbon tetrachloride and subsequently reacting the aromatic-halomethylene intermediate with a nitrile source, such as lithium cyanide, potassium cyanide, or trimethylsilyl cyanide, preferably sodium cyanide. The reaction is carried out at ambient temperatures for about 24 hours, as shown in Step d, to afford the acetonitrile compounds of formula (10), (Larock, R. C., Comprehensive Organic Transformations, copyright 1989, VCH, pp 313; Eur. J. Org. Chem. 1999, 2315-2321).

In Scheme II, Step f, compound of formula (7) is contacted to an appropriate compound of formula (9), this type of compound is known and appreciated in the art (Taylor, Edward C.; Haley, Neil F.; Clemens, Robert J., J. Amer. Chem. Soc., 1981, 7743-7752), to give the compound of formula (2). Typically, the reaction is carried out in an acidic solvent, such as acetic acid and a suitable acid scavenger such as pyridine, or triethylamine. The reaction is carried out at temperatures of about 60° C. to ambient for 4-24 hours.

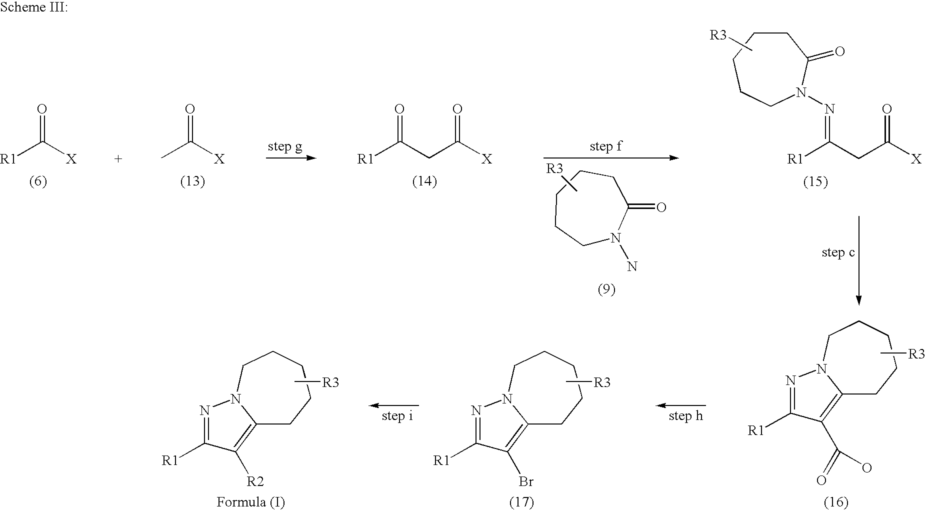

Another variation a skilled artisan would appreciate in the formation of Formula (I) is shown in Scheme III.

Scheme III, Step g, depicts a Claisen condensation of two appropriate substituted carbonyl esters, where X for both compounds of formula (6) and formula (13) is a suitable leaving group as previously described, preferably a C1-C6 alkoxy group. The Claisen condensation is well known and appreciated in the art (March, J., Advanced Organic Chemistry, copyright 1985, John Wiley and Sons, Inc., pp 437-439). The products of formula (14) can be isolated and purified by techniques described above.

In Scheme III, Step f conditions can be applied to a compound of formula (14) with the appropriate compound of formula (9), to give the compound of formula (15). Typically, the reaction is carried out in a suitable solvent such as ethanol, N-methylpyrrolidinone or pyridine with pyridine being the preferred solvent. The reaction is carried out at temperatures of about 60° C. to ambient for 4-24 hours. The products can be isolated and purified by techniques described above.

Step c, as described above, depicts the cyclization of a compound of formula (15) to give an optionally substituted compound of formula (16). Typically, the appropriate compound of formula (15) is reacted with to a suitable base that can form the anion of the hydrazone, sodium hydride being the preferred base in a suitable solvent preferably N,N-dimethylformamide at temperatures of about 0 to 100° C. Optionaly, a hydrolysis of the carboxyl ester of formula (16) can be performed. The products can be isolated and purified by techniques described above.

Step h, depicts the transformation of a carboxylic acid, formula (16), to a halide of formula (17). This transformation is well known and appreciated in the art (Larock, R. C., Comprehensive Organic Transformations, 2^{nd} Ed., copyright 1999, John Wiley & Sons, pp 741-742). The halide of formula (17) can be used as a leaving group in combination with a substituted aryl- or heteroarylboronic acid or ester in the presence of a suitable palladium catalyst, preferably tetrakis(triphenylphosphine)palladium(0), and a suitable base such as potassium carbonate to further give compounds of Formula (I) (Suzuki reaction see: Miryaura, N.; Yanagi, T.; Suzuki, A. The Palladium-Catalyzed Cross Coupling Reaction of Phenylboronic Acid with Haloarenes in the Presence of Bases. Synth. Commun., 1981, 513-518).

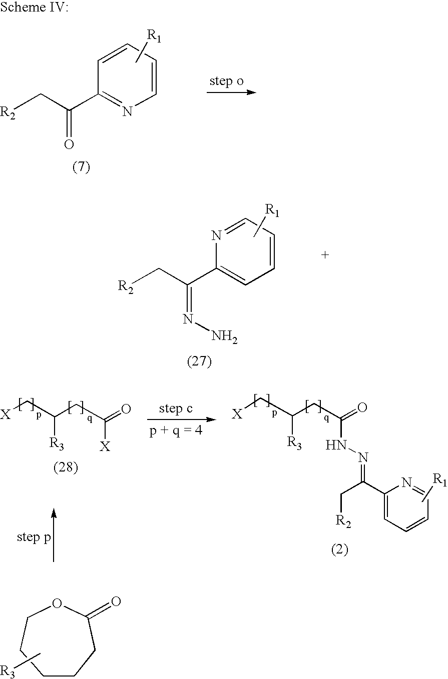

Scheme IV, Step o, depicts a hydrazination of a compound of formula (7) affording a hydrazone compound of formula (27). Typically the reaction is carried out with a suitable source of hydrazine, preferably anhydrous hydrazine in an acidic solution consisting of an alcohol, such as methanol, ethanol, or propanol, and a hydrogen halo acid, preferably hydrogen chloride, is used as the solvent. The product can be isolated and purified by techniques described above. Compounds of formula (28) are commercially available or can be produced by a ring opening of appropriate substituted cyclic-carbonyl esters. Step p depicts these ring openings, which can be accomplished by an acid hydrolysis using reagents such as; hydrogen bromide in acetic acid or trimethyl aluminum to give the corresponding carboxylic acid derivatives to be further transformed to give a compound of formula (28).

Scheme IV, Step c, as previous described, transforms a hydrazone of formula (27) to a hydrazide of formula (2), by acylation with a compound of formula (28). The compound of formula (28) can be an appropriate carboxylic acid derivative, where X can be a leaving group previously described, preferably a halogen, most preferably a chloride, and where p+q equals 4 carbons. The reaction is carried out in the presence of an acid scavenger such as pyridine or triethylamine. The reagents are combined, and products isolated and purified by techniques described above. The conversion of amines to amides by acylation is well known and appreciated in the art (Larock, R. C., Comprehensive Organic Transformations, copyright 1989, VCH, pp 979).

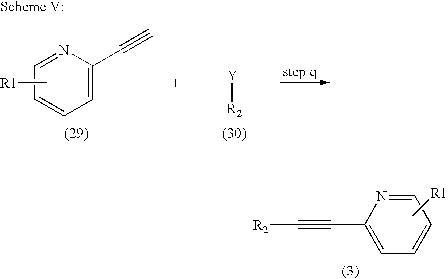

One skilled in the art would appreciate the formation of formula (3) by the palladium-promoted coupling reaction of an alkyne and an aromatic halide. Such a reaction is known and appreciated in the art (Reisch, Johannes; Gunaherath, G. M. Kamal B., J. Heterocycl. Chem., 1993; 1057-1060, Inouye, Masahiko; Miyake, Toshiyuki; Furusyo, Masaru; Nakazumi, Hiroyuki, J. Amer. Chem. Soc., 1995; 12416-12425). For example, in Scheme V, Step q, depicts an appropriately substituted alkyne of formula (29) and a variably substituted compound of formula (30), where R₁ and R₂ are previously described for Formula (I) and where Y can be an appropriate leaving group such as a halide. Typically, the reaction is carried out by combining a compound of formula (30) with a palladium (0) or palladium (II) catalyst as described previously, preferably bis(triphenylphosphine)palladium (II) chloride with a suitable base, such as trialkylamine or pyridine, preferably triethylamine along with a copper(I) halide to facilitate coupling to a compound of formula (29). All reagents are combined in a suitable solvent, typically tetrahydrofuran, toluene or ethylene glycol dimethyl ether, stirred at temperatures of about 0 to 80° C. All products can be isolated and purified by techniques described above.

The skilled artisan will also appreciate that not all of the substituents in the compounds of Formula (I) will tolerate certain reaction conditions employed to synthesize the compounds. These moieties may be introduced at a convenient point in the synthesis, or may be protected and then deprotected as necessary or desired. Furthermore, the skilled artisan will appreciate that in many circumstances, the order in which moieties are introduced-is not critical.

The skilled artisan will appreciate that the compounds of Formula (I) in Methods A, B or C may be formed into acid addition salts using pharmaceutically acceptable acids. The formation of acid-addition salts is well known and appreciated in the art.

The following preparations and examples further illustrate the preparation of compounds of the present invention and should not be interpreted in any way as to limit the scope. Those skilled in the art will recognize that various modifications may be made while not departing from the spirit and scope of the invention. All publications mentioned in the specification are indicative of the level of those skilled in the art to which this invention pertains.

### EXAMPLE 1

### Preparation of 3-quinolin-4-yl-2-pyridin-2-yl-5,6,7,8-tetrahydro-4H-pyrazolo[1,5-a]azepine

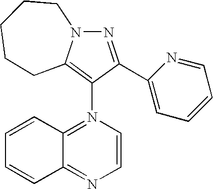 A. Preparation of 6-chloro-hexanoic acid (1-pyridin-2-yl-2-quinolin-4-yl-ethylidene)-hydrazide

Add (1-pyridin-2-yl-2-quinolin-4-yl-ethylidene)-hydrazine (500 mg, 1.9 mmol) to dichloromethane (20 mL). Cool to −78° C. Add pyridine (0.38 mL, 4.75 mmol), next add 6-chloro-hexanoyl chloride (500 mg, 2.85 mmol). Stir for 4 hours. Add methanol (1 mL), then add aqueous saturated ammonium chloride (5 mL). Warm to room temperature. Add dichloromethane (500 mL). Wash with water and brine. Dry the organic solution with sodium sulfate, concentrate in vacuo. Purification by flash column chromatography to provide the subtitled compound (750 mg, 100%) as a pale yellow solid.

¹H NMR (CDCl₃): δ 8.80-8.67 (m, 2H), 8.59-8.50 (m, 1H), 8.25-09 (m, 3H), 7.84-7.72 (m, 2H), 7.70-7.59 (m, 1H), 7.36-7.28 (m, 1H), 6.93-6.88 (m, 1H), 4.88 (s, 2H), 3.59-3.42 (m, 2H), 2.92-2.79 (m, 2H), 2.32-2.18 (m, 1H), 1.89-1.67 (m, 3H), 1.66-1.32 (m, 2H).

B. Preparation of 3-Quinolin-4-yl-2-pyridin-2-yl-5,6,7,8-tetrahydro-4H-pyrazolo[1,5-a]azepine

Add NaH (60% in mineral oil, 200 mg, 5.3 mmol) to a solution of 6-chloro-hexanoic acid (1-pyridin-2-yl-2-quinolin-4-yl-ethylidene)-hydrazide (700 mg, 1.8 mmol) and DMF (11 mL) at 0° C. Heat at 70° C. for 48 h. Quench with aqueous saturated ammonium chloride. Concentrate in vacuo. Take up residue in dichloromethane. Wash with water and brine. Dry the organic solution with sodium sulfate, concentrate in vacuo. Purification by flash column chromatography to provide the title compound (131 mg, 21%) as a brown solid.

Melting Range: 183-185° C.

MS ESI+m/e 341 (M+1)

¹H NMR (CDCl₃): δ 8.92-8.89 (m, 1H), 8.49-8.41 (m, 1H), 8.19-8.09 (m, 1H), 7.71-7.60 (m, 2H), 7.39-7.28 (m, 3H), 7.02-6.91 (m, 2H), 4.52-4.41 (m, 2H), 2.58-2.49 (m, 2H), 1.98-1.79 (m, 4H), 1.63-1.54 (m, 2H).

HPLC: 94.5%, Rₜ=13.40 min.

TLC (SiO₂): R_{f} 0.4 (5:95 methanol/dichloromethane)

### EXAMPLE 2

### Preparation of (7-Methoxy-quinolin-4-yl)-2-pyridin-2-yl-5,6,7,8-tetrahydro-4H-pyrazolo[1,5-a]azepine

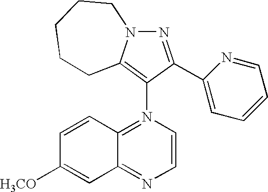 A. Preparation of [2-(7-Methoxy-quinolin-4-yl)-1-pyridin-2-yl-ethylidene]-hydrazine

Add hydrazine monohydrate (2.1 mL, 43.1 mmol) to a solution of 2-(7-methoxy-quinolin-4-yl)-1-pyridin-2-yl-ethanone (2.0 g, 7.2 mmol) in ethanol (35 mL) at 0° C. Add concentrated HCl (0.25 mL). Reflux for 4 hours. Concentrate in vacuo. Take up residue in dichloromethane. Wash with aqueous saturated sodium bicarbonate. Dry the organic solution with sodium sulfate, concentrate in vacuo. Purification by flash column chromatography to provide the subtitled compound (2.0 g, 95%) as a yellow solid.

¹H NMR (CDCl₃): δ 8.69-8.60 (m, 1H), 8.50-8.43 (m, 1H), 8.09-8.01 (m, 2H), 7.71-7.63 (m, 1H), 7.46 (s, 1H), 7.28-7.09 (m, 2H), 6.97-6.89 (m, 1H), 4.37-4.28 (m, 211), 3.99 (s, 3H).

B. Preparation of 6-Chloro-hexanoic acid [2-(7-methoxy-quinolin-4-yl)-1-pyridin-2-yl-ethylidene]-hydrazide

In a similar fashion to Example 1 Step A, react [2-(7-methoxyquinolin-4-yl)-1-pyridin-2-yl-ethylidene]-hydrazine, 1.0 g, 3.4 mmol) with 6-chloro-hexanoyl chloride (1.1 g, 6.8 mmol) to provide the subtitled compound (1.3 g, 90%) as a white solid.

¹H NMR (CDCl₃): δ 8.62-8.58 (m, 1H), 8.52-8.48 (m, 1H), 8.42 (s, 1H), 8.23-8.17 (m, 1H), 8.03-7.93 (m, 1H), 7.46 (s, 1H), 7.32-7.24 (m, 3H), 6.82-6.78 (m, 1H), 4.81 (s, 2H), 3.99 (s, 3H), 3.59-3.49 (m, 2H), 2.89-2.74 (m, 2H), 1.88-1.63 (m, 4H), 1.60-1.52 (2H).

C. Preparation of 3-(7-Methoxy-quinolin-4-yl)-2-pyridin-2-yl-5,6,7,8-tetrahydro-4H-pyrazolo[1,5-a]azepine

In a similar fashion to Example 1, react 6-chloro-hexanoic acid [2-(7-methoxy-quinolin-4-yl)-1-pyridin-2-yl-ethylidene]-hydrazide (1.3 g, 3.1 mmol) with NaH (60% in mineral oil, 370 mg, 9.2 mmol) to provide the title compound (450 mg, 39%) as a white solid.

Melting Range: 127-129° C.

MS ESI+m/e 371 (M+1)

¹H NMR (CDCl₃): δ 8.89-8.82 (m, 1H), 8.52-8.48 (m, 1H), 7.62-7.57 (m, 1H), 7.53 (s, 1H), 7.32-7.24 (m, 1H), 7.12-7.07 (m, 1H), 7.02-6.92 (m, 3H), 4.51-4.42 (m, 2H), 3.99 (s, 3H), 2.59-2.49 (m, 2H), 1.93-1.79 (m, 4H), 1.70-1.56 (m, 2H).

HPLC: >99%, Rₜ=14.39 min.

TLC (SiO₂): R_{f} 0.4 (5:95 methanol/dichloromethane)

### EXAMPLE 3

### Preparation of 4-(2-Pyridin-2-yl-5,6,7,8-tetrahydro-4H-pyrazolo[1,5-a]azepin-3-yl)-quinolin-7-ol

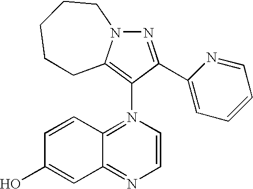

Add ethanethiol (1.0 mL, 13.5 mmol) to a solution of 3-(7-methoxy-quinolin-4-yl)-2-pyridin-2-yl-5,6,7,8-tetrahydro-4H-pyrazolo[1,5-a]azepine (Example 2, 250 mg, 670 mmol) in DMF (8 mL). Slowly add NaH (60% in mineral oil, 500 mg, 13.5 mmol). Stir at room temperature for 20 min. Heat at 80° C. for 6 h. Cool to room temperature. Quench with saturated aqueous ammonium chloride. Concentrate in vacuo. Triturate with water. Purify by flash column chromatography to provide the title compound (80 mg, 38%) as a yellow solid.

MS ESI+m/e 357 (M+1)

¹H NMR (CD₃OD): δ 8.71-8.60 (m, 1H), 8.32-8.24 (m, 1H), 7.60-7.54 (m, 1H), 7.51-7.47 (m, 1H), 7.32-7.24 (m, 2H), 7.14-7.07 (m, 2H), 6.97-6.88 (m, 1H), 4.51-4.42 (m, 2H), 2.68-2.50 (m, 2H), 1.99-1.79 (m, 4H), 1.70-1.56 (m, 2H).

HPLC: >99%, Rₜ=11.69 min.

TLC (SiO₂): R_{f} 0.28 (5:1:95 methanol/concentrated aqueous ammonium hydroxide/dichloromethane)

### EXAMPLE 4

### Preparation of 3-[7-(2-Morpholin-4-yl-ethoxy)-quinolin-4-yl]-2-pyridin-2-yl-5,6,7,8-tetrahydro-4H-pyrazolo[1,5-a]azepine

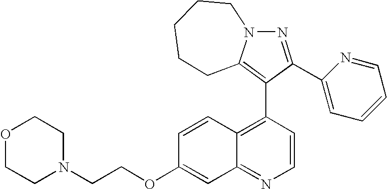

Add NaH (60% in mineral oil, 20 mg, 460 mmol) to a solution of 4-(2-pyridin-2-yl-5,6,7,8-tetrahydro-4H-pyrazolo[1,5-a]azepin-3-yl)-quinolin-7-ol (Example 3, 55 mg, 150 mmol), 4-(2-chloroethyl)morpholine hydrochloride (56 mg, 300 mmol) in DMF (5 mL). Stir at room temperature for 20 min. Heat at 50° C. for 24 hours. Cool to room temperature. Quench with saturated aqueous ammonium chloride. Concentrate in vacuum. Take up residue in dichloromethane. Wash with water and brine. Dry the organic solution with sodium sulfate, concentrate in vacuum. Purify by flash column chromatography to provide the title compound (60 mg, 86%) as a white solid.

Melting Range: 157-159° C.

MS ESI+m/e 470 (M+1)

¹H NMR (CDCl₃): δ 8.87-8.81 (m, 1H), 8.51-8.44 (m, 1H), 7.60-7.54 (m, 1H), 7.48-7.42 (m, 1H), 7.37-7.28 (m, 2H), 7.16-7.10 (m, 1H), 7.07-6.95 (m, 3H), 4.57-4.46 (m, 2H), 4.31-4.22 (m, 2H), 3.79-3.70 (m, 4H), 2.93-2.83 (m, 2H), 2.64-2.56 (m, 2H), 2.55-2.49 (m, 2H), 1.99-1.79 (m, 4H), 1.70-1.56 (m, 2H).

HPLC: >99%, Rₜ=12.60 min.

TLC (SiO₂): R_{f} 0.32 (5:1:95 methanol/concentrated aqueous ammonium hydroxide/dichloromethane)

The compounds disclosed herein were tested by the following protocols for TGF-β inhibition, as described below in the protocol description.

### TGF-β Receptor I Purification and In Vitro Kinase Reactions

For TGF-β Type I (RIT204D) Receptors:

The 6×-HIS tagged cytoplasmic kinase domain of each receptor was expressed and purified from Sf9 insect cell lysates as briefly described below:

Cell pellets after 48-72 hours of infection were lysed in lysis buffer (LB: 50 mM Tris pH 7.5, 150 mM NaCl, 50 mM NaF, 0.5% NP40 with freshly added 20 mM β-mercaptoethanol, 10 mM imidazole, 1 mM PMSF, 1×EDTA-free Complete Protease Inhibitor(Boehringer Mannheim).

Cell lysates were clarified by centrifugation and 0.45 uM filtered prior to purification by Ni/NTA affinity chromatography (Qiagen).

Chromatography Protocol:

Equilibrate with 10 CV of LB, load sample, wash with 10 CV RIPA buffer (50 mM Tris pH 7.5, 150 mM NaCl, 1% NP40, 1 mM EDTA, 0.25% sodium deoxycholate, added fresh 20 mM β-mercaptoethanol, 1 mM PMSF), wash with 10 CV LB, wash with 10 CV 1×KB (50 mM Tris pH 7.5, 150 mM NaCl, 4 mM MgCl₂, 1 mM NaF, 2 mM β-mercaptoethanol), elute with a linear gradient of 1×KB containing 200 mM Imidazole.

Both enzymes were approximately 90% pure and had autophosphorylation activity.

Reactions: 170-200 nM enzyme in 1×KB, compound dilution series in 1×KB/16% DMSO (20 μM to 1 nM final concentration with 4% DMSO final concentration), reactions started by adding ATP mix (4 uM ATP/1 uCi ³³P-γ-ATP final concentrations) in 1×KB.

Reactions were incubated at 30° C. for 1 hour. Reactions were stopped and quantitated using standard TCA/BSA precipitation onto Millipore FB glass fiber filter plates and by liquid scintillation counting on a MicroBeta JET.

Representative compounds of the current invention which inhibit the TGF-β Type I (RIT204D) receptor kinase domain with IC₅₀ values <20 μM are listed in Table I.

### TGF-β Receptor I TABLE I

### Compound Name

a. 3-quinolin-4-yl-2-pyridin-2-yl-5,6,7,8-tetrahydro-4H-pyrazolo[1,5-a]azepine.
b. (7-Methoxy-quinolin-4-yl)-2-pyridin-2-yl-5,6,7,8-tetrahydro-4H-pyrazolo[1,5-a]azepine
c. 4-(2-Pyridin-2-yl-5,6,7,8-tetrahydro-4H-pyrazolo[1,5-a]azepin-3-yl)-quinolin-7-ol
d. 3-[7-(2-Morpholin-4-yl-ethoxy)-quinolin-4-yl]-2-pyridin-2-yl-5,6,7,8-tetrahydro-4H-pyrazolo[1,5-a]azepine

Conditions “characterized by enhanced TGF-β activity” include those wherein TGF-β synthesis is stimulated so that TGF-β is present at increased levels or wherein TGF-β latent protein is undesirably activated or converted to active TGF-β protein or wherein TGF-β receptors are upregulated or wherein the TGF-β protein shows enhanced binding to cells or extracellular matrix in the location of the disease. Thus, in either case “enhanced activity” refers to any condition wherein the biological activity of TGF-β is undesirably high, regardless of the cause.

A number of diseases have been associated with TGF-β1 over production. Inhibitors of TGF-β intracellular signaling pathway are useful treatments for fibroproliferative diseases. Specifically, fibroproliferative diseases include kidney disorders associated with unregulated TGF-β activity and excessive fibrosis including glomerulonephritis (GN), such as mesangial proliferative GN, immune GN, and crescentic GN. Other renal conditions include diabetic nephropathy, renal interstitial fibrosis, renal fibrosis in transplant patients receiving cyclosporin, and HIV-associated nephropathy. Collagen vascular disorders include progressive systemic sclerosis, polymyositis, scleroderma, dermatomyositis, eosinophilic fascitis, morphea, or those associated with the occurrence of Raynaud's syndrome. Lung fibroses resulting from excessive TGF-β activity include adult respiratory distress syndrome, idiopathic pulmonary fibrosis, and interstitial pulmonary fibrosis often associated with autoimmune disorders, such as systemic lupus erythematosus and scleroderma, chemical contact, or allergies. Another autoimmune disorder associated with fibroproliferative characteristics is rheumatoid arthritis.

Eye diseases associated with a fibroproliferative condition include retinal reattachment surgery accompanying proliferative vitreoretinopathy, cataract extraction with intraocular lens implantation, and post glaucoma drainage surgery are associated with TGF-β1 overproduction.

Fibrotic diseases associated with TGF-β1 overproduction can be divided into chronic conditions such as fibrosis of the kidney, lung and liver and more acute conditions such as dermal scarring and restenosis (Chamberlain, J. Cardiovascular Drug Reviews, 19(4):329-344). Synthesis and secretion of TGF-β1 by tumor cells can also lead to immune suppression such as seen in patients with aggressive brain or breast tumors (Arteaga, et al. (1993) J. Clin. Invest. 92:2569-2576). The course of Leishmanial infection in mice is drastically altered by TGF-β1 (Barral-Netto, et al. (1992) Science 257:545-547). TGF-β1 exacerbated the disease, whereas TGF-β1 antibodies halted the progression of the disease in genetically susceptible mice. Genetically resistant mice became susceptible to Leishmanial infection upon administration of TGF-β1.

The profound effects of TGF-β1 on extracellular matrix deposition have been reviewed (Rocco and Ziyadeh (1991) in Contemporary Issues in Nephrology v. 23, Hormones, autocoids and the kidney. ed. Jay Stein, Churchill Livingston, N.Y. pp. 391-410; Roberts, et al. (1988) Rec. Prog. Hormone Res. 44:157-197) and include the stimulation of the synthesis and the inhibition of degradation of extracellular matrix components. Since the structure and filtration properties of the glomerulus are largely determined by the extracellular matrix composition of the mesangium and glomerular membrane, it is not surprising that TGF-β1 has profound effects on the kidney. The accumulation of mesangial matrix in proliferative glomerulonephritis (Border, et al. (1990) Kidney Int. 37:689-695) and diabetic nephropathy (Mauer, et al. (1984) J. Clin. Invest. 74:1143-1155) are clear and dominant pathological features of the diseases. TGF-β1 levels are elevated in human diabetic glomerulosclerosis (advanced neuropathy) (Yamamoto, et al. (1993) Proc. Natl. Acad. Sci. 90:1814-1818). TGF-β1 is an important mediator in the genesis of renal fibrosis in a number of animal models (Phan, et al. (1990) Kidney Int. 37:426; Okuda, et al. (1990) J. Clin. Invest. 86:453). Suppression of experimentally induced glomerulonephritis in rats has been demonstrated by antiserum against TGF-β1 (Border, et al. (1990) Nature 346:371) and by an extracellular matrix protein, decorin, which can bind TGF-β1 (Border, et al. (1992) Nature 360:361-363).

Too much TGF-β1 leads to dermal scar-tissue formation. Neutralizing TGF-β1 antibodies injected into the margins of healing wounds in rats have been shown to inhibit scarring without interfering with the rate of wound healing or the tensile strength of the wound (Shah, et al. (1992) Lancet 339:213-214). At the same time there was reduced angiogenesis, reduced number of macrophages and monocytes in the wound, and a reduced amount of disorganized collagen fiber deposition in the scar tissue.

TGF-β1 may be a factor in the progressive thickening of the arterial wall which results from the proliferation of smooth muscle cells and deposition of extracellular matrix in the artery after balloon angioplasty. The diameter of the restenosed artery may be reduced 90% by this thickening, and since most of the reduction in diameter is due to extracellular matrix rather than smooth muscle cell bodies, it may be possible to open these vessels to 50% simply by reducing extensive extracellular matrix deposition. In uninjured pig arteries transfected in vivo with a TGF-β1 gene, TGF-β1 gene expression was associated with both extracellular matrix synthesis and hyperplasia (Nabel, et al. (1993) Proc. Natl. Acad. Sci. USA 90:10759-10763). The TGF-β1 induced hyperplasia was not as extensive as that induced with PDGF-BB, but the extracellular matrix was more extensive with TGF-β1 transfectants. No extracellular matrix deposition was associated with FGF-1 (a secreted form of FGF) induced hyperplasia in this gene transfer pig model (Nabel (1993) Nature 362:844-846).

There are several types of cancer where TGF-β1 produced by the tumor may be deleterious. MATLyLu rat prostate cancer cells (Steiner and Barrack (1992) Mol. Endocrinol 6:15-25) and MCF-7 human breast cancer cells (Arteaga, et al. (1993) Cell Growth and Differ. 4:193-201) became more tumorigenic and metastatic after transfection with a vector expressing the mouse TGF-β1. TGF-β1 has been associated with angiogenesis, metastasis and poor prognosis in human prostate and advanced gastric cancer (Wikstrom, P., et al. (1998) Prostate 37: 19-29; Saito, H. et al. (1999) Cancer 86: 1455-1462). In breast cancer, poor prognosis is associated with elevated TGF-β (Dickson, et al. (1987) Proc. Natl. Acad. Sci. USA 84:837-841; Kasid, et al. (1987) Cancer Res. 47:5733-5738; Daly, et al. (1990) J. Cell Biochem. 43:199-211; Barrett-Lee, et al. (1990) Br. J Cancer 61:612-617; King, et al. (1989) J. Steroid Biochem. 34:133-138; Welch, et al. (1990) Proc. Natl. Acad. Sci. USA 87:7678-7682; Walker, et al. (1992) Eur. J. Cancer 238:641-644) and induction of TGF-β1 by tamoxifen treatment (Butta, et al. (1992) Cancer Res. 52:4261-4264) has been associated with failure of tamoxifen treatment for breast cancer (Thompson, et al. (1991) Br. J. Cancer 63:609-614). Anti TGF-β1 antibodies inhibit the growth of MDA-231 human breast cancer cells in athymic mice (Arteaga, et al. (1993) J. Clin. Invest. 92:2569-2576), a treatment which is correlated with an increase in spleen natural killer cell activity. CHO cells transfected with latent TGF-β1 also showed decreased NK activity and increased tumor growth in nude mice (Wallick, et al. (1990) J. Exp. Med. 172:1777-1784). Thus, TGF-β secreted by breast tumors may cause an endocrine immune suppression. High plasma concentrations of TGF-β1 have been shown to indicate poor prognosis for advanced breast cancer patients (Anscher, et al. (1993) N. Engl. J. Med. 328:1592-1598). Patients with high circulating TGF-β before high dose chemotherapy and autologous bone marrow transplantation are at high risk for hepatic veno-occlusive disease (15-50% of all patients with a mortality rate up to 50%) and idiopathic interstitial pneumonitis (40-60% of all patients). The implication of these findings is 1) that elevated plasma levels of TGF-β1 can be used to identify at risk patients and 2) that reduction of TGF-β1 could decrease the morbidity and mortality of these common treatments for breast cancer patients.

Many malignant cells secrete transforming growth factor-β (TGF-β), a potent immunosuppressant, suggesting that TGF-β production may represent a significant tumor escape mechanism from host immunosurveillance. Establishment of a leukocyte sub-population with disrupted TGF-β signaling in the tumor-bearing host offers a potential means for immunotherapy of cancer. A transgenic animal model with disrupted TGF-β signaling in T cells is capable of eradicating a normally lethal TGF-β overexpressing lymphoma tumor, EM4 (Gorelik and Flavell, (2001) Nature Medicine 7(10): 1118-1122). Down regulation of TGF-β secretion in tumor cells results in restoration of immunogenicity in the host, while T-cell insensitivity to TGF-β results in accelerated differentiation and autoimmunity, elements of which may be required in order to combat self-antigen-expressing tumors in a tolerized host. The immunosuppressive effects of TGF-β have also been implicated in a subpopulation of HIV patients with lower than predicted immune response based on their CD4/CD8 T cell counts (Garba, et al. J. Immunology (2602) 168: 2247-2254). A TGF-β neutralizing antibody was capable of reversing the effect in culture, indicating that TGF-β signaling inhibitors may have utility in reversing the immune suppression present in this subset of HIV patients.

During the earliest stages of carcino genesis, TGF-β1 can act as a potent tumor suppressor and may mediate the actions of some chemopreventive agents. However, at some point during the development and progression of malignant neoplasms, tumor cells appear to escape from TGF-β-dependent growth inhibition in parallel with the appearance of bioactive TGF-β in the microenvironment. The dual tumor suppression/tumor promotion roles of TGF-β have been most clearly elucidated in a transgenic system overexpressing TGF-β in keratinocytes. While the transgenics were more resisitant to formation of benign skin lesions, the rate of metastatic conversion in the transgenics was dramatically increased (Cui, et al (1996) Cell 86(4):531-42). The production of TGF-β1 by malignant cells in primary tumors appears to increase with advancing stages of tumor progression. Studies in many of the major epithelial cancers suggest that the increased production of TGF-β by human cancers occurs as a relatively late event during tumor progression. Further, this tumor-associated TGF-β provides the tumor cells with a selective advantage and promotes tumor progression. The effects of TGF-β on cell/cell and cell/stroma interactions result in a greater propensity for invasion and metastasis. Tumor-associated TGF-β may allow tumor cells to escape from immune surveillance since it is a potent inhibitor of the clonal expansion of activated lymphocytes. TGF-β has also been shown to inhibit the production of angiostatin. Cancer therapeutic modalities such as radiation therapy and chemotherapy induce the production of activated TGF-β in the tumor, thereby selecting outgrowth of malignant cells that are resistant to TGF-β growth inhibitory effects. Thus, these anticancer treatments increase the risk and hasten the development of tumors with enhanced growth and invasiveness. In this situation, agents targeting TGF-β-mediated signal transduction might be a very effective therapeutic strategy. The resistance of tumor cells to TGF-β has been shown to negate much of the cytotoxic effects of radiation therapy and chemotherapy and the treatment-dependent activation of TGF-β in the stroma may even be detrimental as it can make the microenvironment more conducive to tumor progression and contributes to tissue damage leading to fibrosis. The development of a TGF-β signal transduction inhibitors is likely to benefit the treatment of progressed cancer alone and in combination with other therapies.

The compounds are useful for the treatment of cancer and other disease states influenced by TGF-β by inhibiting TGF-β in a patient in need thereof by administering said compound(s) to said patient. TGF-β would also be useful against atherosclerosis (T. A. McCaffrey: TGF-βs and TGF-β Receptors in Atherosclerosis: Cytokine and Growth Factor Reviews 2000, 11, 103-114) and Alzeheimer's (Masliah, E.; Ho, G.; Wyss-Coray, T.: Functional Role of TGF-β in Alzheimer's Disease Microvascular Injury: Lessons from Trangenic Mice: Neurochemistry International 2001, 39, 393-400) diseases.

### Pharmaceutical Compositions

The compositions of the present invention are therapeutically effective amounts of the TGF-β antagonists, noted above. The composition may be formulated with common excipients, diluents or carriers, and compressed into tablets, or formulated elixirs or solutions for convenient oral administration or administered by intramuscular intravenous routes. The compounds can be administered transdermally and maybe formulated as sustained release dosage forms and the like.

The method of treating a human patient according to the present invention includes administration of the TGF-β antagonists. The TGF-β antagonists are formulated into formulations which may be administered by the oral and rectal routes, topically, parenterally, e.g., by injection and by continuous or discontinuous intra-arterial infusion, in the form of, for example, tablets, lozenges, sublingual tablets, sachets, cachets, elixirs, gels, suspensions, aerosols, ointments, for example, containing from 1 to 10% by weight of the active compound in a suitable base, soft and hard gelatin capsules, suppositories, injectable solutions and suspensions in physiologically acceptable media, and sterile packaged powders adsorbed onto a support material for making injectable solutions. Advantageously for this purpose, compositions may be provided in dosage unit form, preferably each dosage unit containing from about 5 to about 500 mg (from about 5 to 50 mg in the case of parenteral or inhalation administration, and from about 25 to 500 mg in the case of oral or rectal administration) the compounds. Dosages from about 0.5 to about 300 mg/kg per day, preferably 0.5 to 20 mg/kg, of active ingredient may be administered although it will, of course, readily be understood that the amount of the compound actually to be administered will be determined by a physician, in the light of all the relevant circumstances including the condition to be treated, the choice of compound to be administered and the choice of route of administration and therefore the above preferred dosage range is not intended to limit the scope of the present invention in any way.

The formulations useful for separate administration of the TGF-β antagonists will normally consist of at least one compound selected from the compounds specified herein mixed with a carrier, or diluted by a carrier, or enclosed or encapsulated by an ingestible carrier in the form of a capsule, sachet, cachet, paper or other container or by a disposable container such as an ampoule. A carrier or diluent may be a solid, semi-solid or liquid material which serves as a vehicle, excipient or medium for the active therapeutic substance. Some examples of the diluents or carrier which may be employed in the pharmaceutical compositions of the present invention are lactose, dextrose, sucrose, sorbitol, mannitol, propylene glycol, liquid paraffin, white soft paraffin, kaolin, fumed silicon dioxide, microcrystalline cellulose, calcium silicate, silica, polyvinylpyrrolidone, cetostearyl alcohol, starch, modified starches, gum acacia, calcium phosphate, cocoa butter, ethoxylated esters, oil of theobroma, arachis oil, alginates, tragacanth, gelatin, syrup, methyl cellulose, polyoxyethylene sorbitan monolaurate, ethyl lactate, methyl and propyl hydroxybenzoate, sorbitan trioleate, sorbitan sesquioleate and oleyl alcohol and propellants such as trichloromonofluoromethane, dichlorodifluoromethane and dichlorotetrafluoroethane. In the case of tablets, a lubricant may be incorporated to prevent sticking and binding of the powdered ingredients in the dies and on the punch of the tableting machine. For such purpose there may be employed for instance aluminum, magnesium or calcium stearates, talc or mineral oil.

Preferred pharmaceutical forms of the present invention are capsules, tablets, suppositories, injectable solutions, creams and ointments. Especially preferred are formulations for inhalation application, such as an aerosol, for injection, and for oral ingestion.

1-3. (canceled)

4. A compound of the formula: 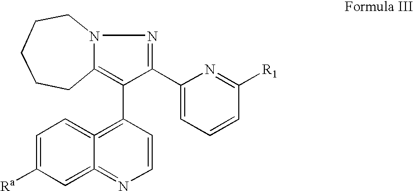
wherein
R¹ is hydrogen or methyl;
R^{a} is hydroxy; (C1-C4)alkoxy; or —O(CH₂)₂N-morpholino;
and the pharmaceutically acceptable salts thereof.

5. A compound according to claim 4 selected from the group consisting of:
a. 3-quinolin-4-yl-2-pyridin-2-yl-5,6,7,8-tetrahydro-4H-pyrazolo[1,5-a]azepine.
b. (7-Methoxy-quinolin-4-yl)-2-pyridin-2-yl-5,6,7,8-tetrahydro-4H-pyrazolo[1,5a]azepine.
c. 4-(2-Pyridin-2-yl-5,6,7,8-tetrahydro-4H-pyrazolo[1,5-a]azepin-3-yl)-quinolin-7-ol.
d. 3-[7-(2-Morpholin-4-yl-ethoxy)-quinolin-4-yl]-2-pyridin-2-yl-5,6,7,8-tetrahydro-4H-pyrazolo[1,5-a]azepine.

6. A method of treating cancer, fibrosis, restenosis, wound healing, HIV infection, alzheimers' disease and/or atherosclerosis comprising administering to a patient in need thereof a compound of the formula: 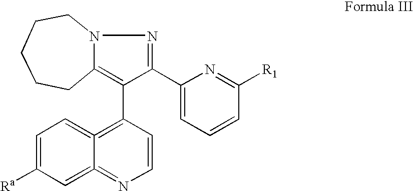
wherein
R¹ is hydrogen or methyl;
R^{a} is hydroxy; (C1-C4)alkoxy; or —(CH₂)₂N-morpholino;
and the pharmaceutically acceptable salts thereof.

7. A pharmaceutical formulation comprising a compound of the formula: 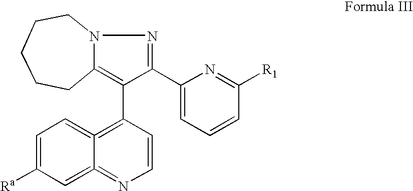
wherein
R¹ is hydrogen or methyl;
R^{a} is hydroxy; (C1-C4)alkoxy; or —O(CH₂)₂N-morpholino;
and the pharmaceutically acceptable salts thereof, in combination with a pharmaceutically acceptable diluent, carrier, or excipient.
